(19) United States
(12) Patent Application Publication
(10) Pub. No.: US 2007/0255110 A1
(43) Pub. Date: Nov. 1, 2007

(54) Operative vaginal speculum

(75) Inventors: Joseph R. Wax, Oxford, ME (US); Andrea Tetreau, Westbrook, ME (US)

Correspondence Address: CHRIS A. CASEIRO, VERRILL DANA, LLP, ONE PORTLAND SQUARE, PORTLAND, ME, 04112-0586, US

(73) Assignee: Maine Medical Center, Portland, ME (US)

(21) Appl. No.: 11/413,278

(22) Filed: Apr. 28, 2006

(51) Int. Cl. A61B 1/32

(52) U.S. Cl. 600/223

(57) ABSTRACT

A speculum for performing gynecological procedures. The speculum includes a first structure, which includes a fixed blade member, a second structure, which includes a movable blade member, and a third structure to join the first and second structures together. The first structure includes a light source holder for an associated light source. The speculum includes a hinge, which may be located on either side of the speculum. These features facilitate improved visualization of, and access to, the genital tract. Such enhanced visualization and access reduces the likelihood of patient discomfort. The fixed blade member and the second blade member may be formed with cutaways to enhance visibility and instrument placement through the speculum.

### BACKGROUND OF THE INVENTION

1. Field of the Invention

The present invention relates to a vaginal speculum. More particularly, the present invention relates to a vaginal speculum having an associated light source.

2. Description of the Prior Art

Specula are universally needed for the routine and specialized care of all women. Specula currently being used for this purpose include singly-hinged metallic specula that do not accommodate an internal light source, which therefore means that when one of these specula is used, proper lighting must be supplied from an external source. Significant limitations accompany the use of external lighting, however. For one, the body of the external light source can obstruct the professional's vision of the genital tract. Such decreased visualization can slow the professional's progress, and thereby increase the time needed to perform the procedure. This is undesirable because time spent undergoing a procedure can directly affect patient well-being, as longer procedures generally are more painful and more discomforting than are shorter ones. Furthermore, depending on the specific nature of the procedure being performed, obstructed vision increases the likelihood that the professional will make an error which harms the patient, such as making an improperly placed or overly deep incision.

Another drawback to specula of this variety is that they are not designed for single-use, but instead are meant to be re-used many times with sterilization occurring between each use. Sterilization, however, can be costly and time-consuming. More significantly, if sterilization is not done properly, blood borne pathogens or other harmful biological agents from one patient can survive the sterilization process and be transmitted to another.

Other specula being used include those that are plastic, double-hinged, and light source-accommodating. Unlike metallic specula, these plastic specula are intended only for single-use, but like metal specula, they too are considerably flawed. In particular, their bulky double-hinge makes it difficult for the professional to manipulate instruments, such as scissors, forceps, and probes, that are commonly used in conjunction with the speculum. Also, like the external light source, this double-hinge can obstruct the professional's vision of the genital tract. For these reasons, some of the problems associated with specula requiring an external light source, which are namely prolongation of the procedure and increased risk of harm to the patient, also are characteristic of double-hinged specula.

What is needed therefore is a vaginal speculum which is meant for single-use, and which has both a single-hinge and a light source arranged to allow unobstructed visualization of, and unencumbered access to, the genital tract.

### SUMMARY OF THE INVENTION

It is an object of the present invention to provide a vaginal speculum which allows optimal visualization of, and free access to, the genital tract. Features of the present invention that facilitate better visualization of, and freer access to, the genital tract, include an internally-contained light source, a hinge positioned out of the direct line of sight, and a pair of blade members arranged for optimal viewing therebetween. Further facilitating this goal is the optional choice to position the hinge on either side of the blade pair, to accommodate the left-handedness or right-handedness of the user.

It is another object of the present invention to provide a vaginal speculum having blade members which may be pivoted toward and away from each other about a single axis by using one mechanism, and which may be vertically moved toward and away from each other while the blades are held parallel, or essentially parallel, to each other by using a second mechanism. No matter which of these two mechanisms are used to position the blade members, it is an object of the present invention to provide a means of reversibly fixing the position of the blade members by providing locking elements for both mechanisms.

It is another object of the present invention to provide a disposable vaginal speculum, which is intended to be used only once. The disposable nature of the present invention eliminates the need for sterilization, which can be expensive, time-consuming, and most importantly, when not done properly, can lead to blood-borne pathogens being transmitted to a patient in the course of even the most routine gynecological procedure.

The details of one or more examples related to the invention are set forth in the accompanying drawings and the description below. Other features, objects, and advantages of the invention will be apparent from the description and the drawings.

### BRIEF DESCRIPTION OF THE DRAWINGS

FIG. 1 shows a side view of the speculum of the present invention;

FIG. 2 shows a rear perspective view of the speculum of the present invention from the left side;

FIG. 3 shows a rear perspective view of the speculum of the present invention from the right side;

FIG. 4 shows a top view of the speculum of the present invention;

FIG. 5 shows a perspective view of the first structure of the present invention;

FIG. 6 shows a rear view of the first structure of the present invention;

FIG. 7 shows a side view of the first structure of the present invention;

FIG. 8 shows a front view of the first structure of the present invention;

FIG. 9 shows a top view of the first structure of the present invention;

FIG. 10 shows a perspective view of the second structure of the present invention;

FIG. 11 shows a top view of the second structure of the present invention;

FIG. 12 shows a side view of the second structure of the present invention;

FIG. 13 shows a rear view of the second structure of the present invention;

FIG. 14 shows a perspective view of the third structure of the present invention;

FIG. 15 shows a top view of a right-sided version of the third structure of the present invention;

FIG. 16 shows a bottom view of a right-sided version of the third structure of the present invention;

FIG. 17 shows a side view of the third structure of the present invention;

FIG. 18 shows a bottom view of the third structure of the present invention;

FIG. 19 shows a top view of a second embodiment of the shape of either or both of the first and second blade members of the present invention.

### DETAILED DESCRIPTION OF THE PREFERRED EMBODIMENTS OF THE INVENTION

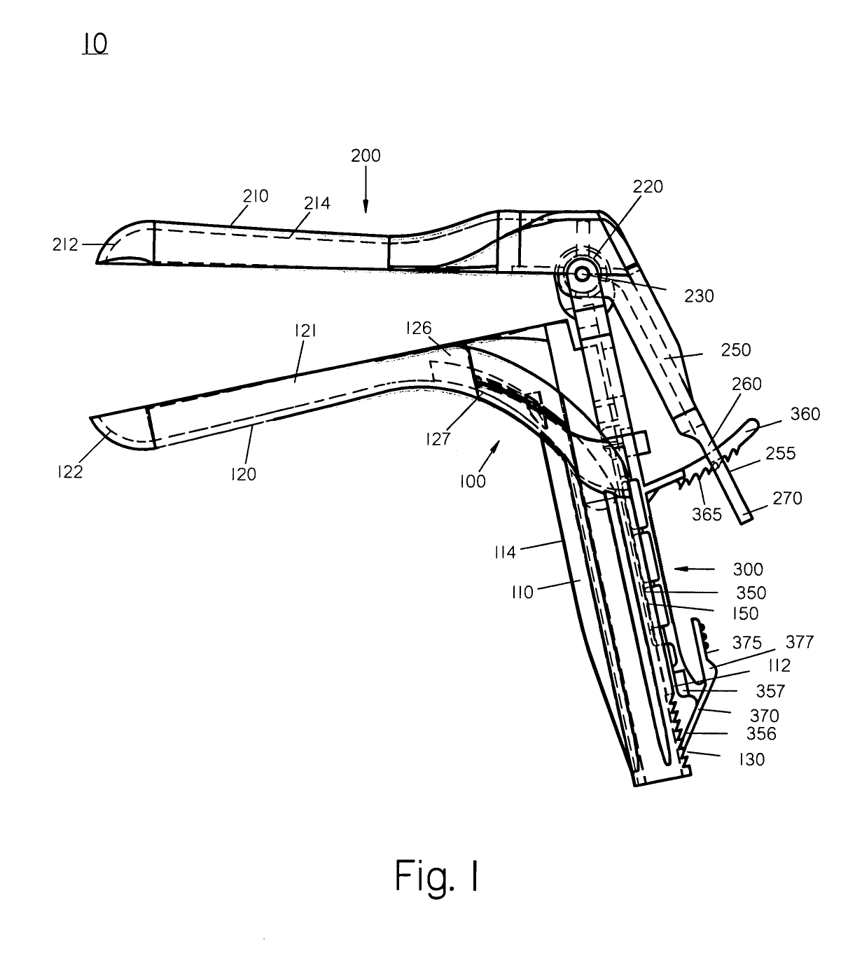

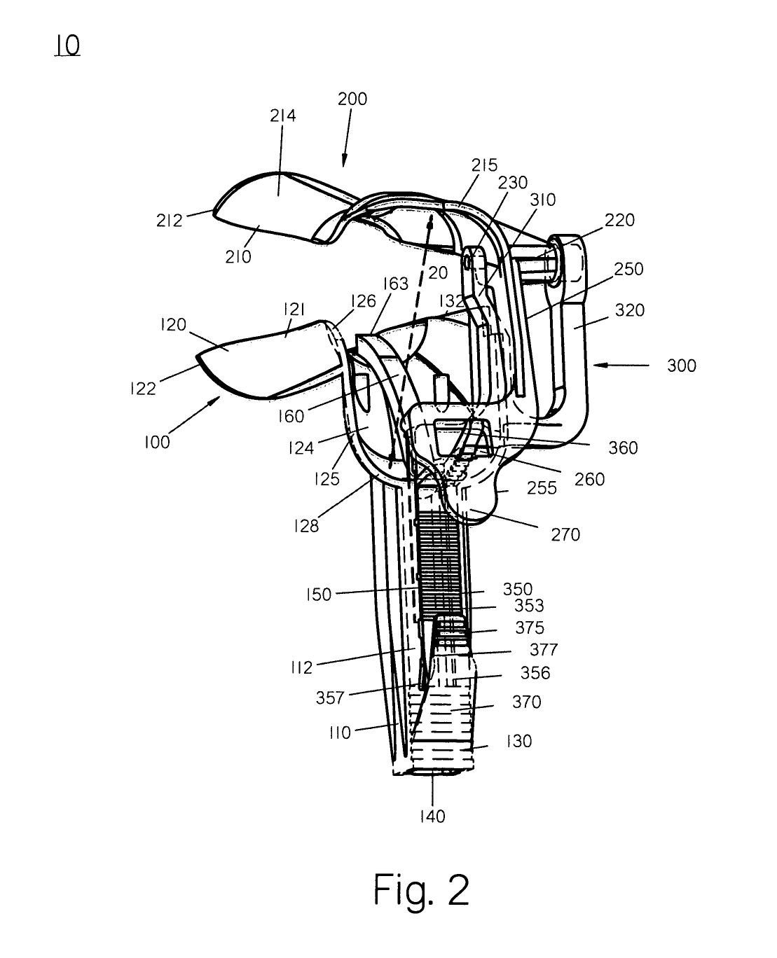

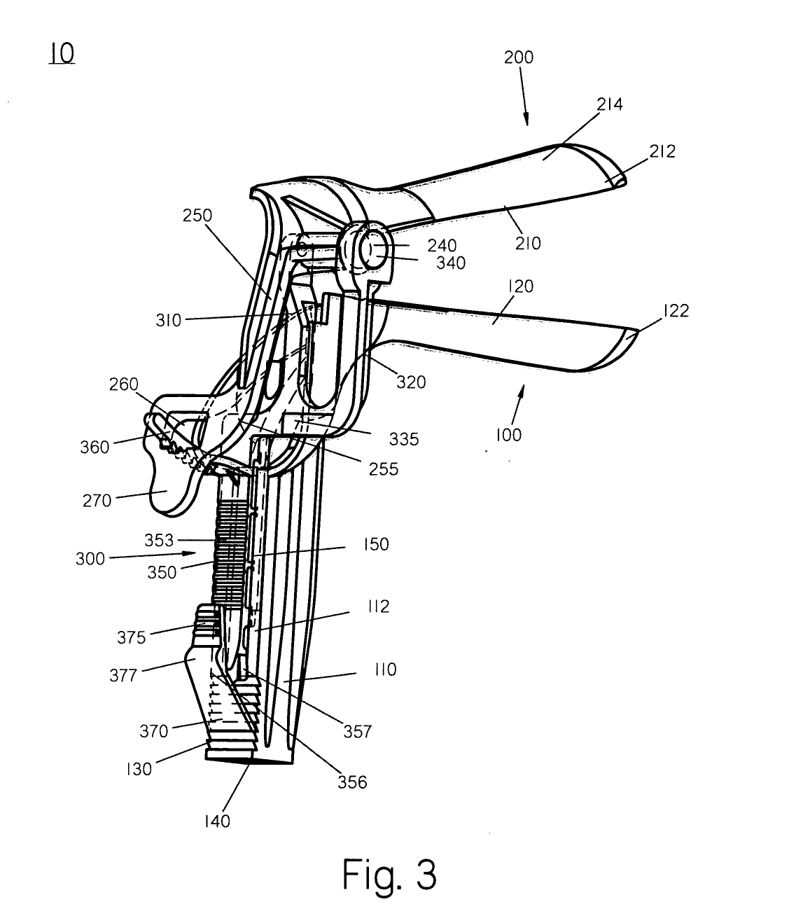

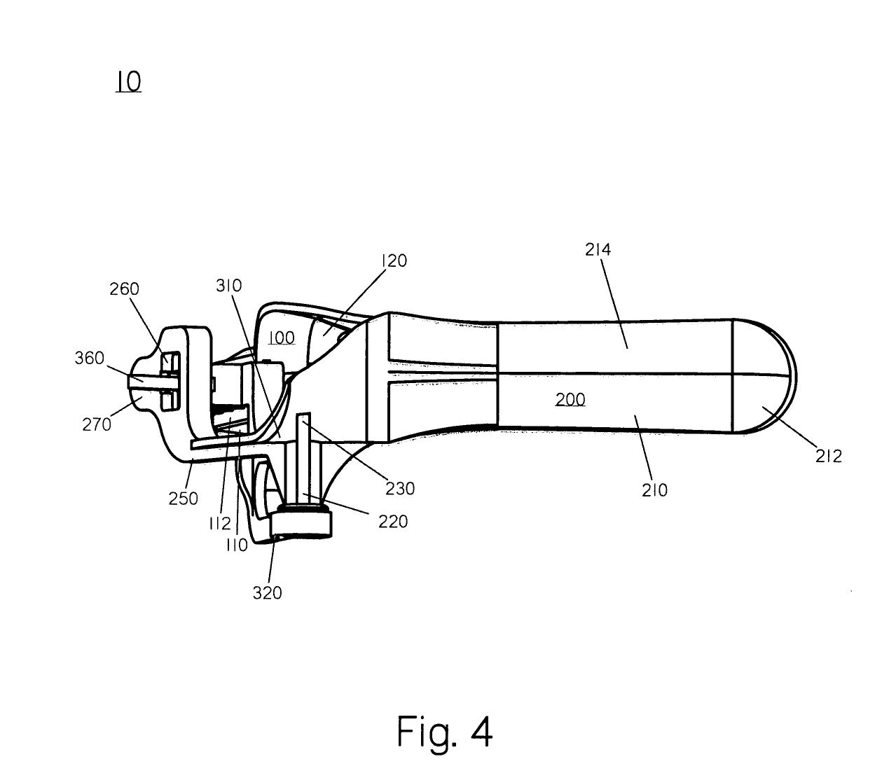

As shown in FIGS. 1-4, the present invention is a vaginal speculum 10 including three structures, which are a first structure 100, a second structure 200, and a third structure 300. The first structure 100 is a fixed first blade structure, the second structure 200 is a movable second blade structure, and the third structure 300 fixes and moves the second structure 200 with respect to the first structure 100. The first structure 100, second structure 200, and third structure 300 work in combination to form an easy-to-use speculum 10 with an improved line-of-sight and convenient lighting.

The first structure 100 includes a holder, to be described herein, for retaining a light source therein. The third structure 300 joins the first structure 100 to the second structure 200 in a way that enables pivoting movement of the second structure to and away from the first structure without effect on the internal positioning of the light source when retained in the first structure 100. The third structure 300 also joins the first structure 100 and the second structure 200 together so that the second structure 200 may be spaced away from the first structure 100 to change the size of the opening formed by the position of the second structure 200 with respect to the first structure 100.

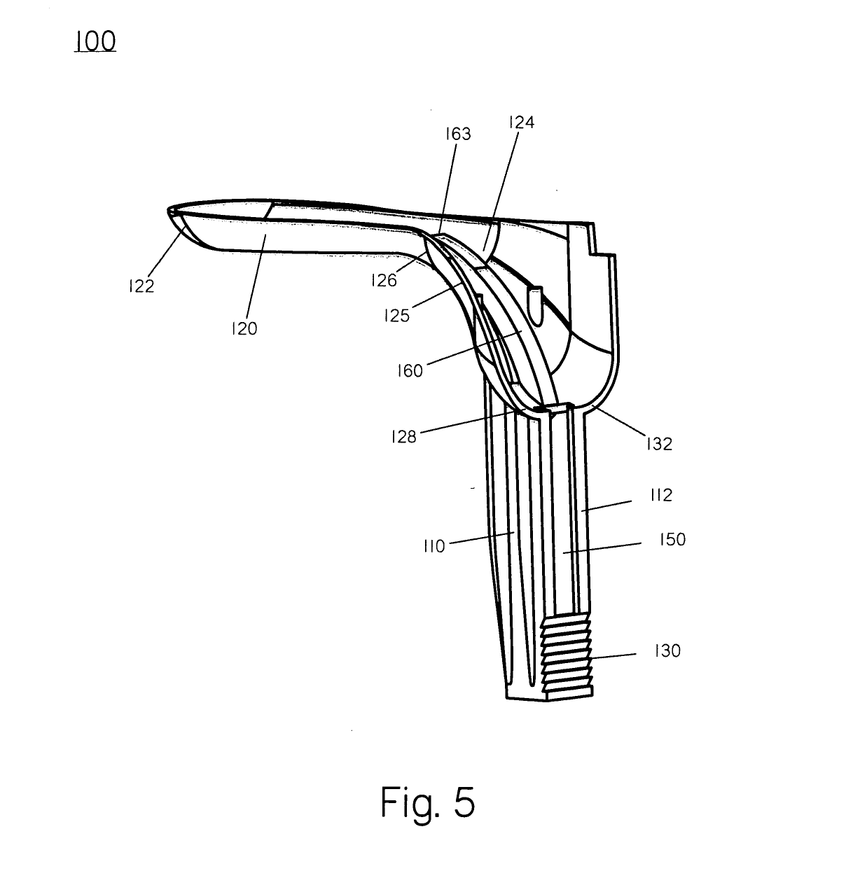

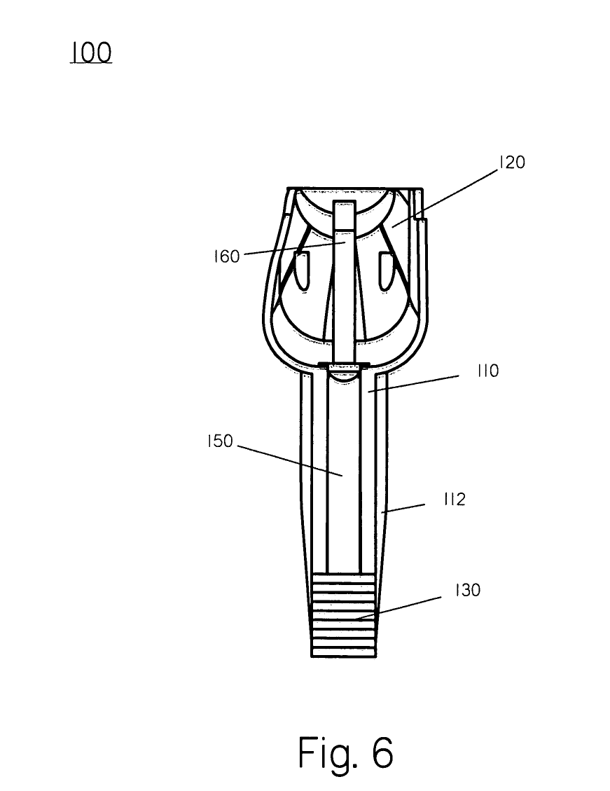

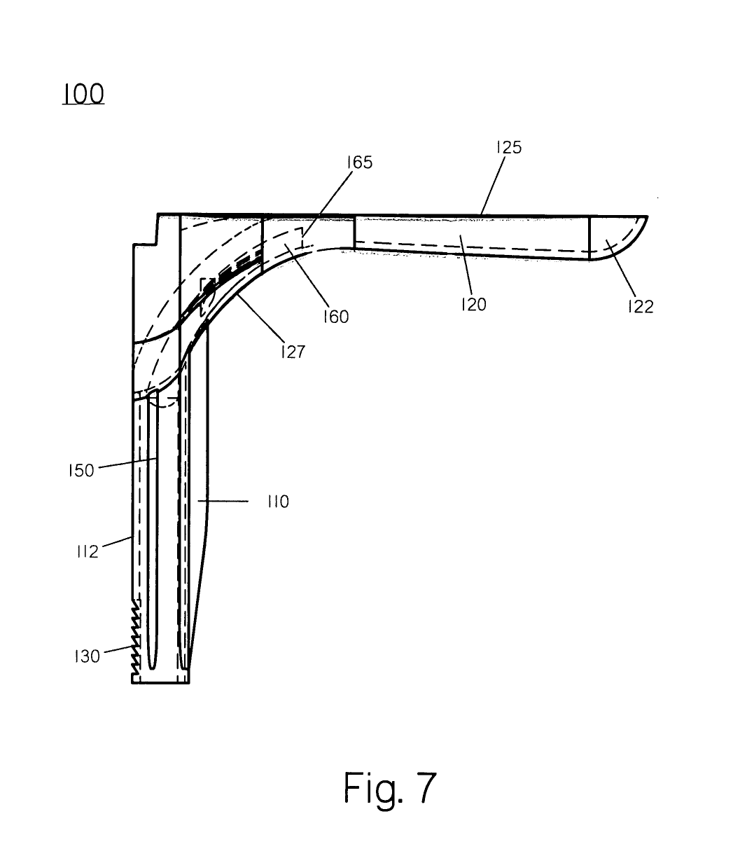

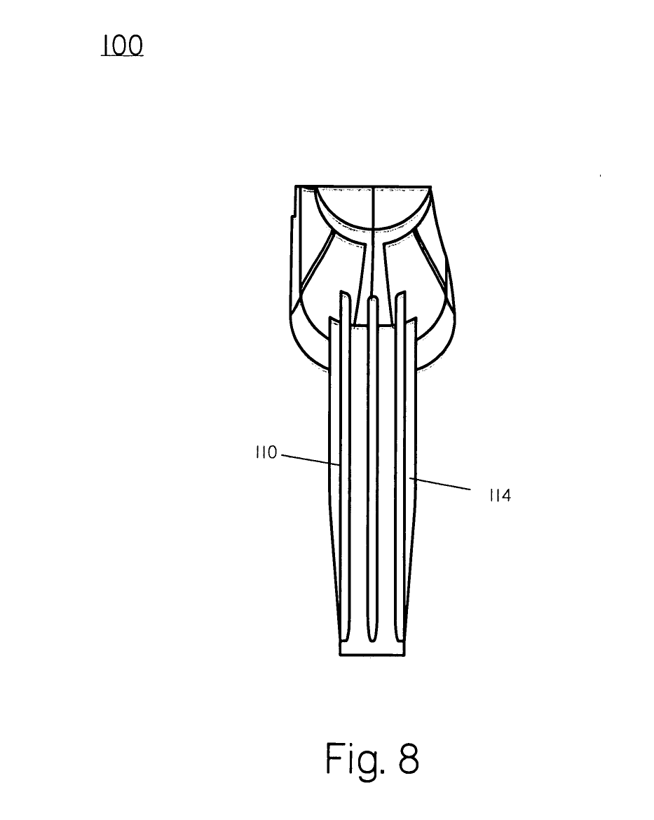

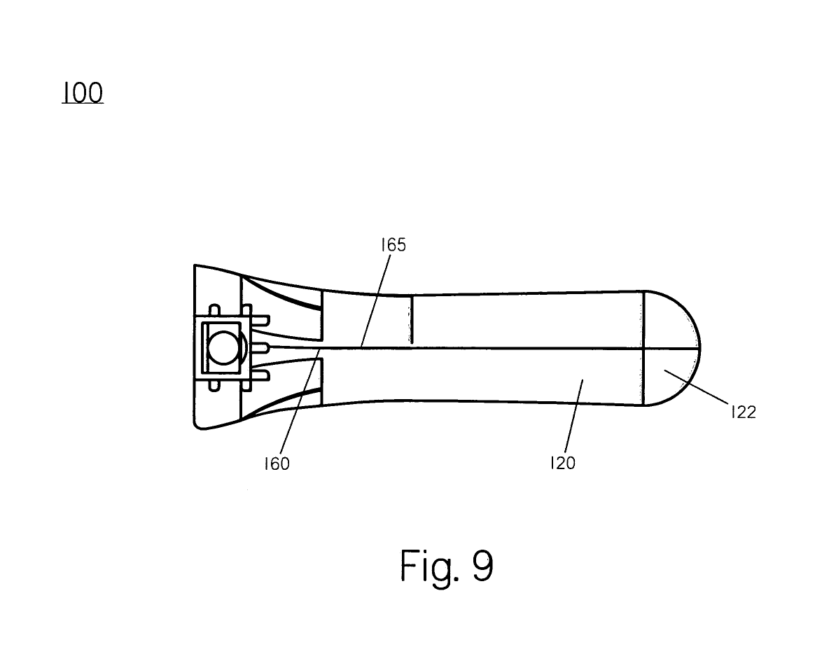

In addition to being shown in context as part of the speculum 10 in FIGS. 1-4, the first structure 100 is shown individually in FIGS. 5-9. As illustrated in FIGS. 1-9, the first structure 100 includes a handle 110 and a first blade member 120. The first blade member 120 and the handle 110 may be connected together, or formed integrally with one another, such that they are oriented to form an essentially right angle. Of course, it is to be understood that their orientation with respect to one another is not so limited and other angles may be formed. The handle 110 includes an anterior face 112, a posterior face 114, a linear-position fixer 130, a light source port 140, and a track 150. The first blade member 120 includes a primary section 121, a tip 122, an inner face 124, a lip 125, a transition section 126 including a first side transition section 128 and a second side transition section 132 and a light source holder 160. The transition section 126 defines an interface between the primary section 121 and the handle 110. The light source holder 160 extends from the light source port 140 of the handle 110, through the transition section 126, and ends with a light source holder end 163 in the primary section 121.

The lip 125 of the first blade member 120 runs along its entire perimeter from the tip 122 to the transition section 126. On the second side transition section 132 of the first blade member 120, the lip 125 remains substantially on a single plane from the tip 122 through the primary section 121 to near the end of the second side transition section 132. On the first side transition section 128 of the first blade member 120, the lip 125 remains substantially on a single plane in parallel with the plane of the lip 125 on the second side transition section 132 from the tip 122 through the primary section 120 up to the transition section 126. At about region 127, where the first blade member 120 transitions to handle 110, the lip 125 begins to slope sharply downward when viewed with the speculum 10 in an upright position with the handle 110 below the first blade member 120. The cutaway resulting from the formation of the transition section 126 on the first side transition section 128 serves to create a lower portion of a wide mouth 20 between the first structure 100 and the second structure 200 of the speculum 10. The wide mouth 20 provides the professional using the speculum 10 with more room to manipulate gynecological instruments, such as forceps, scissors and probes, than does a narrower mouth. A narrower mouth would be formed if the first side transition section 128 mirrored the arrangement of the second side transition 132. The present invention may be formed in that manner, but providing the wide mouth 20 is preferred.

The light source holder 160 is located along the inner face 124 of the first blade member 120. The end 163 of the light source holder 160, which faces the tip 112 of the second blade, has an opening 165, or alternatively a transparent face, through which light may pass. The light source port 140 provides an entry to the light source holder 160 for insertion of a light source therein. The handle 110 may be hollowed to form at least a portion of the light source holder 160. Alternatively, the handle 110 may include a conduit of similar dimensions to that portion of the light source holder 160 located in the transition section 126 of the first blade member 120. In this arrangement, a light source may be fed up through the port 140 until the light-emitting end thereof essentially reaches the opening 165. Once positioned within the light source holder 160, the light source may be permanently or removably retained therein.

One example of a light source is a bulb, such as an 8 volt halogen bulb. The bulb may be powered by battery or by electrical wiring which may or may not be coupled to an external power supply.

The track 150 is preferably positioned on the anterior face 112 of the handle 110. It is arranged to receive therein a portion of the third structure 300 in a manner to be described herein. The linear position fixer 130 also couples to the third structure 300 in a manner to be described herein. The linear position fixer 130 may be any sort of component or design suitable for allowing the linear movement along, and fixing thereon, of another device. For the purpose of describing the preferred embodiment of the present invention, the linear position fixer 130 is a plurality of grooves 130 located on the anterior face 112 of the handle 110 below the track 150.

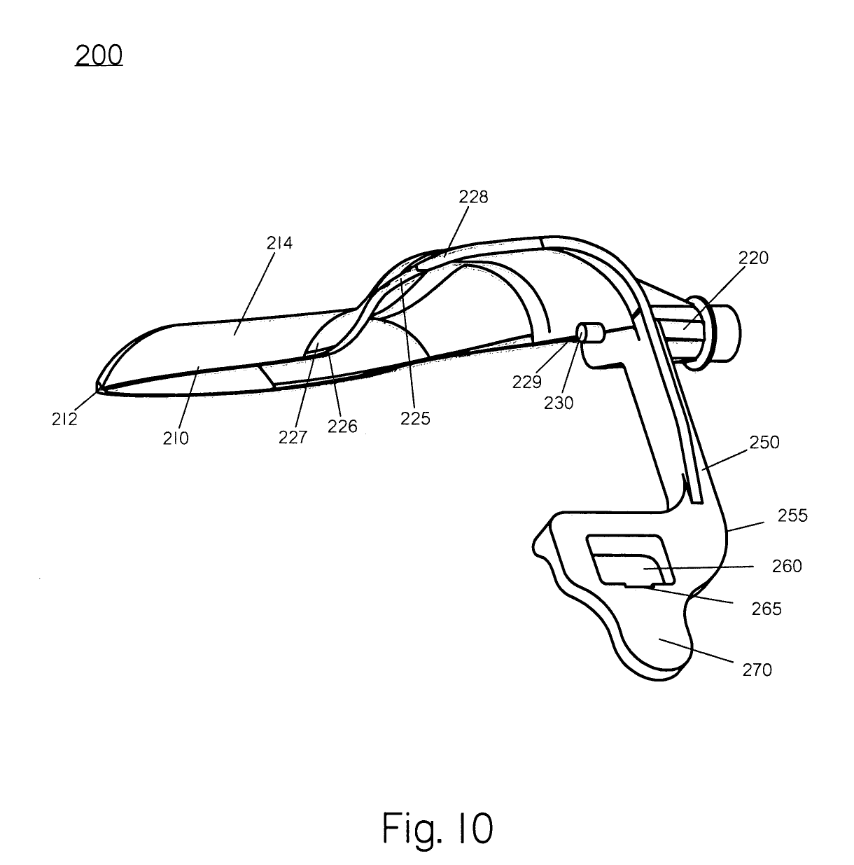

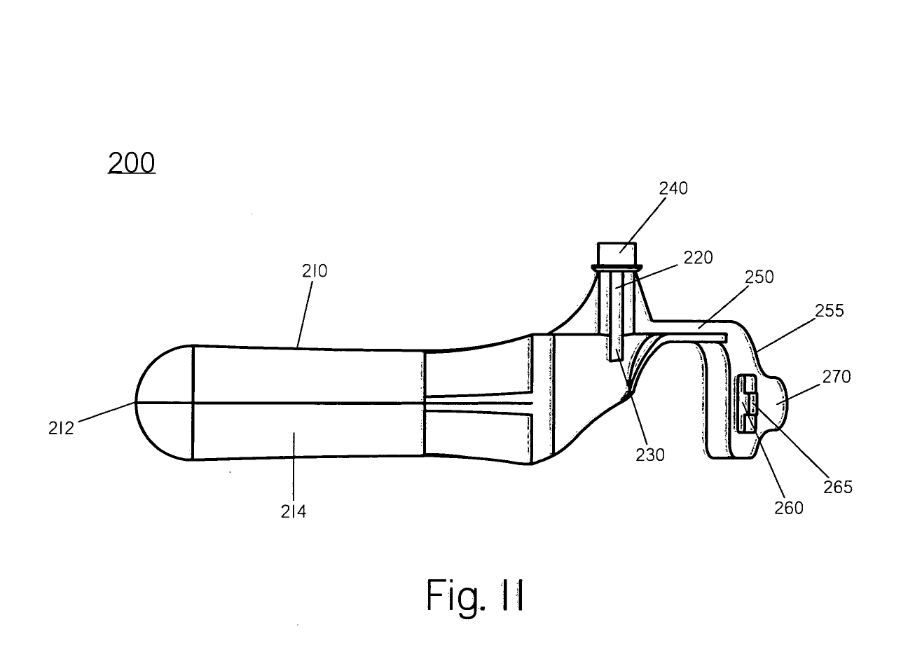

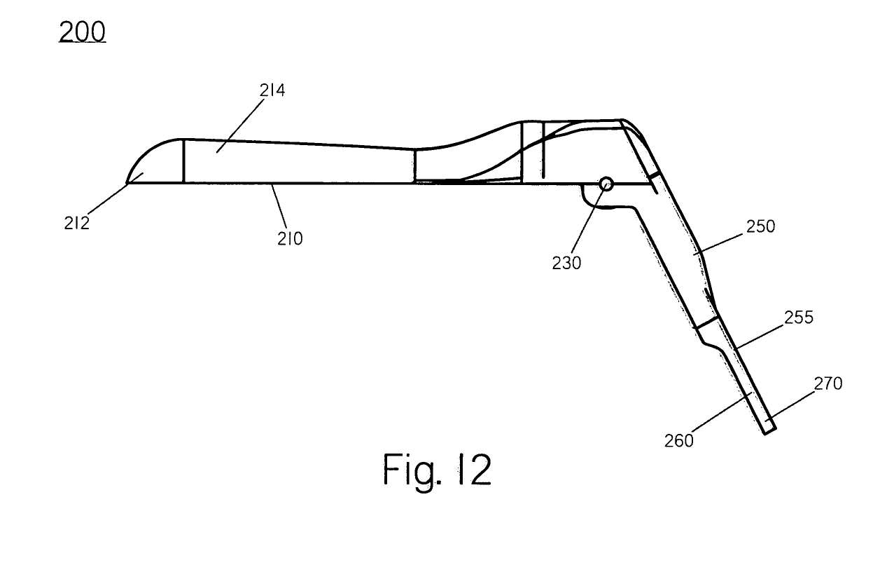

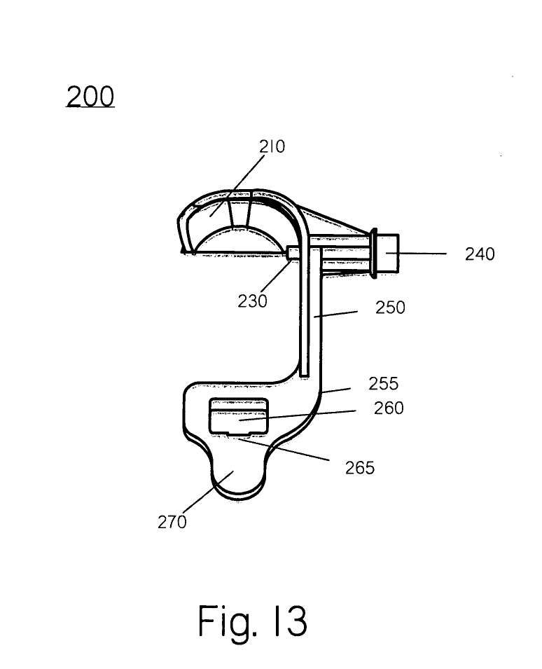

In addition to being shown in context as part of the speculum 10 in FIGS. 1-4, the second structure 200 is shown individually in FIGS. 10-13. As illustrated in FIGS. 1-4 and 10-13, the second structure 200 includes a second blade member 210, a tip 212, a primary section 214, a hinge 220, a lip 225, a transition section 226 including a first side transition section 228 and a second side transition section 229, a structural member 250, and a coupling element 255.

The lip 225 of the second blade member 210 runs along its entire perimeter from the tip 212 to the transition section 226. On the second side transition section 229 of the second blade member 210, the lip 225 remains substantially on a single plane from the tip 212 through the primary section 214 to near the end of the second side transition section 229. On the first side transition section 228 of the second blade member 210, the lip 225 remains substantially on a single plane in parallel with the plane of the lip 225 on the second side transition section 229 from the tip 212 through the primary section 214 up to the transition section 226. At about region 227, the lip 225 begins to slope sharply upward when viewed with the speculum 10 in an upright position with the handle 110 below the first blade member 120. The cutaway resulting from the formation of the transition section 226 on the first side transition section 228 serves to create an upper portion of the wide mouth 20 between the first structure 100 and the second structure 200 of the speculum 10. A narrower mouth would be formed if the first side transition section 228 mirrored the arrangement of the second side transition 229. The present invention may be formed in that manner, but providing the wide mouth 20 is preferred. The lip 125 of the first blade member 120 and the lip 225 of the second blade member 210 oppose one another when the speculum 10 is complete, in a substantially mirror image of one another.

With continuing reference to FIGS. 1-4 and 10-13, the hinge 220 of the second structure 200 extends outwardly to the side from the axial orientation of the first blade member 120 and the second blade member 210. The hinge 220 has a first hinge end 230 which extends from the hinge 220 and toward the center of the second blade member 210 at the transition section 226, and a second hinge end 240 which extends away from the second blade member 210. The hinge 220, the first hinge end 230 and second hinge end 240 may be one integral piece. Alternatively, one or both of the hinge ends 230 and 240 may be separate from the hinge 220. For example, the hinge 220 may contain a hole through which a pin is inserted such that the ends of the pin serve as hinge ends 230 and 240.

The hinge 220 is preferably compact, spaced away from the viewing area and/or otherwise formed to allow the professional to better see within the speculum 10 to the vaginal tract. The hinge 220 may be formed such that, when the speculum 10 is viewed from the back end toward tips 112 and 212, it may be located to the right or the left of the second blade member 210. Although all of the Figures herein which show the hinge 220, show it as being located to the right of the first blade member 120 when viewed, again, from the rear of the speculum 10, it is also contemplated that the hinge 220 may be located on the opposite, or left, side of the speculum 10. The configuration of the speculum with the hinge 220 on the right side allows a right-handed professional to use the speculum in the right hand and manipulate instruments with the left, and vice-versa when the hinge 220 is positioned on the left side of the speculum 10.

The second structure 200 further includes as the primary structural member 250 a vertical pivoting arm 250, which extends angularly from the second blade member 210. The vertical pivoting arm 250 is fabricated to provide structural integrity and rigidity of the second blade member 210 as that member is moved when the speculum 10 is in use. The vertical pivoting arm 250 provides a structural transition from the hinge 220 to the coupling element 255. The coupling element 255 connects the second structure 200 to the third structure 300. The coupling element 255 includes a port 260 and a pivot flange 270. The port 260 is designed to receive and releasably retain therein an element of the third structure 300. The port 260 preferably includes a notch 265 which is located at the region of the port 260 which is closest to the pivot flange 270. The pivot flange 270 provides a location for the professional to place a thumb or finger and force the pivoting of the second blade member 210 about the hinge 220 in relation to the first blade member 120.

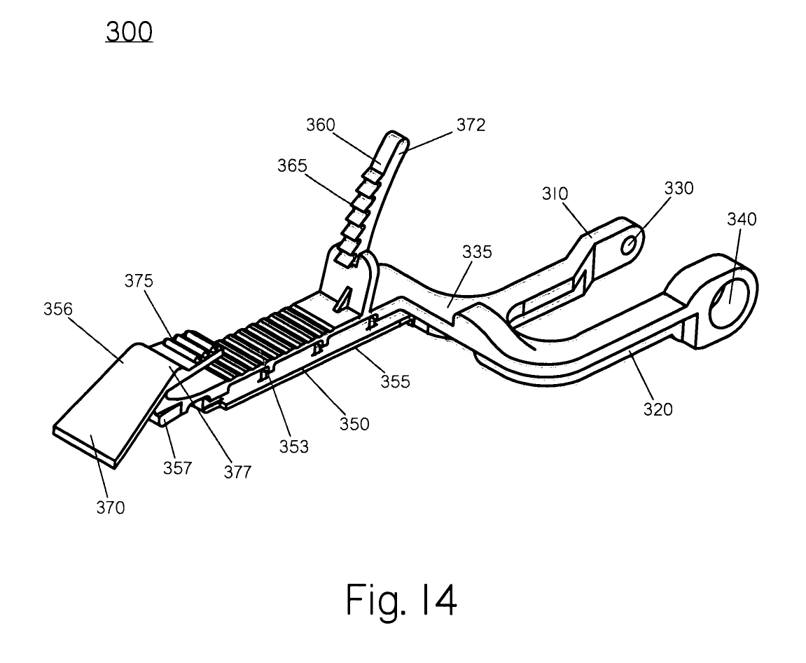

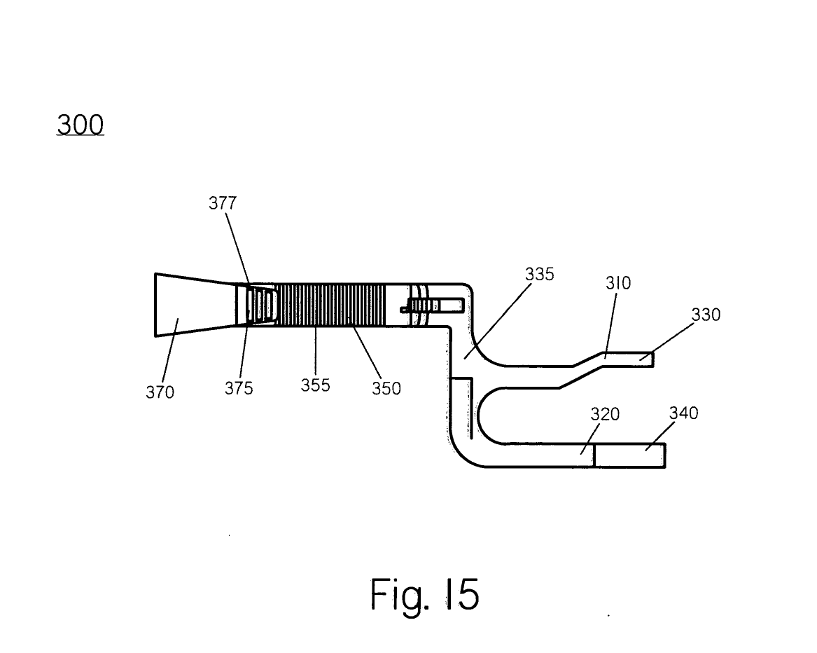

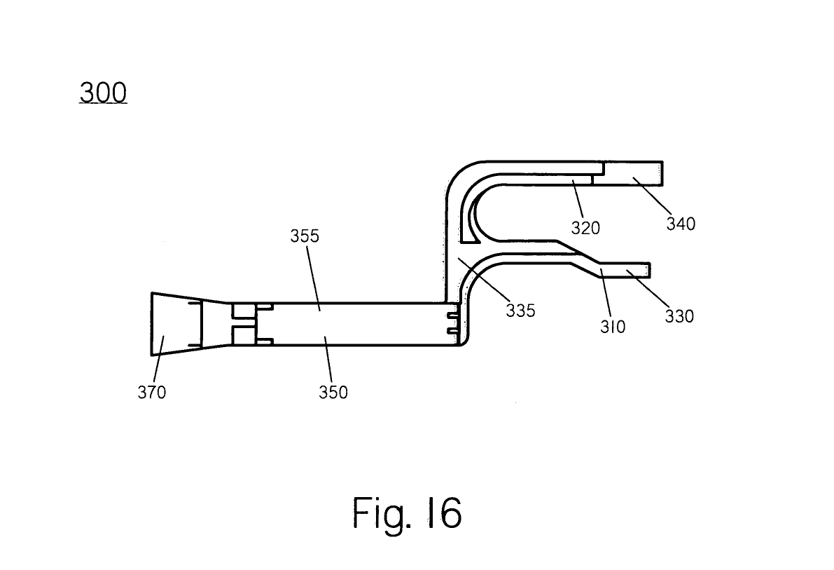

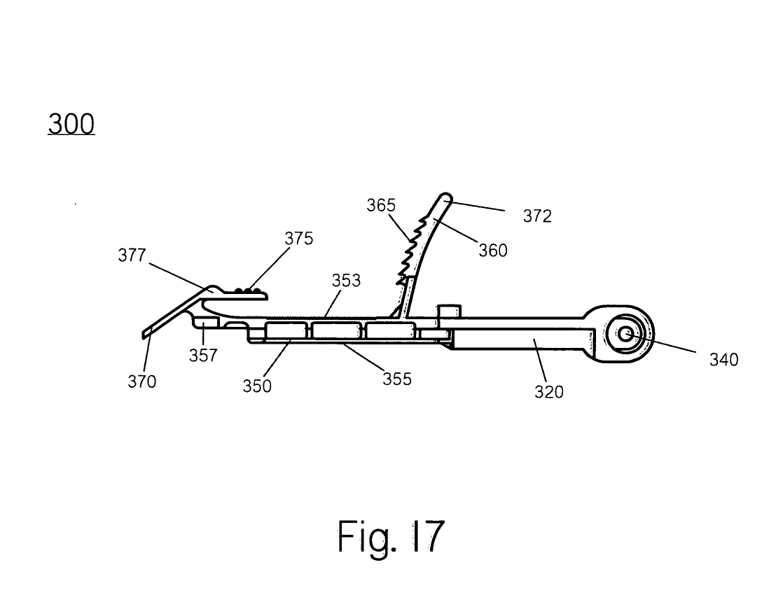

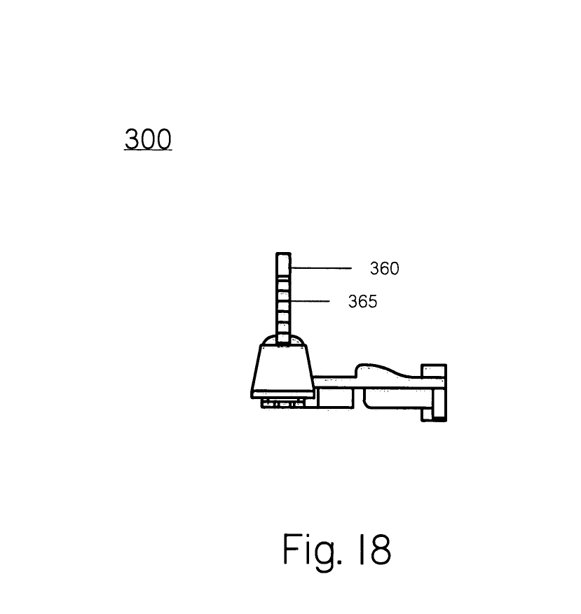

In addition to being shown in context as part of the speculum 10 in FIGS. 1-4, the third structure 300 is shown individually in FIGS. 14-18. The third structure 300 includes a first hinge-retaining arm 310, a second hinge-retaining arm 320, a transition leg 335, a linear position sliding section 350 with an anterior surface 353, a posterior surface 355 and a bottom end 357, a linear position-locking arm 356, and a pivot position-locking arm 360. The first hinge-retaining arm 310 includes a first hinge end holding port 330 for retaining therein the first hinge end 230 of the second structure 200. The second hinge-retaining arm 320 includes a second hinge end holding port 340 for retaining therein the second hinge end 240 of the second structure 200. The first hinge-retaining arm 310 and the second hinge-retaining arm 320 operate together to pivotally connect the second structure 200 to the third structure 300.

The first hinge-retaining arm 310 and the second hinge-retaining arm 320 are connected together and to the linear position sliding section 350 by the transition leg 335. The transition leg 335 is preferably fabricated and/or formed with sufficient structural characteristics to permit operation of the speculum 10 as desired without structural failure. All components may be formed integrally, or separately and fixed together. The linear position sliding section 350 is arranged to be fixedly or removably retained within the track 150 of the handle 110. It includes means on the anterior surface 353 thereof to enable linear movement of the third structure 300, and thus the second structure 200 in relation to the handle and thus, the second blade member 210 in relation to the first blade member 120. In the embodiment of the third structure 300 shown in the figures, that means for enabling linear movement is a series of grooves arranged to contact and correspond to groove retaining elements of the track 150. Selectable linear positioning of the linear position sliding section 350 in the track 150 sets the parallel spacing of the second blade member 210 with respect to the first blade member 120.

The linear position sliding section 350 also provides a support structure for the linear position-locking arm 356 and the pivot position-locking arm 360. The linear position-locking arm 356 located at one end of the linear position sliding section 350 extends outwardly from the linear position sliding section 350 and includes an angular locking clip 370, a depressible thumb pad 375 and a lower base 377. The depressible thumb pad 375 is essentially parallel to the anterior surface 353 of the linear position sliding section 350. The angular locking clip 370 extends from the lower base 377 of the depressible thumb pad 375 and in a direction toward and below the bottom end 357 of the linear position sliding section 350. The locking clip 370 is arranged to be releasably affixable in one of the plurality of grooves 130 of the handle 110. The locking clip 370 allows the professional to fix parallel spacing of the second blade member 210 with respect to the first blade member 120 without applying constant pressure to the thumb pad 375.

The pivot position-locking arm 360 includes a plurality of locking teeth 365 for releasably retaining thereon the coupling element 255 of the second structure 200, preferably by contacting the notch 265 thereof. The pivot position-locking arm 360 further preferably includes a release tab 372 for separating the locking teeth 365 from the coupling element 255. The pivot position-locking arm 360 allows the professional to pivot the second blade member 210 with respect to the position of the first blade member 120 and fix it in place at the selectable opening size. The professional is thus not required to maintain constant pressure on the pivot flange 270 to maintain the opening.

As shown in FIGS. 1-4 and previously stated, the first structure 100, second structure 200 and third structure 300 of the speculum 10 are interconnected. Specifically, the third structure 300 is directly engaged with both the first structure 100 and the second structure 200. By being directly engaged with the third structure 300, the first structure 100 and the second structure 200 are indirectly engaged with each other. Furthermore, at no point are the first structure 100 and second structure 200 directly connected to each other.

The speculum 10 of the present invention may be used to continuously exert pressure on the walls of a vagina for the purpose of holding the vagina open. For this reason, the speculum 10 must be sturdy. In a preferred embodiment of the present invention, the speculum 10 is made of one or more sturdy plastics or plastic composites. Exemplary plastic materials which may be used to construct the speculum 10 include, but are not limited to, polypropylene, polyethylene, and polystyrene, and any composite of more than one of these plastics. The speculum 10, however, is not limited being made of plastic. In an alternative embodiment of the present invention, the speculum 10 may be made in whole or in part of one or more metals or metal alloys. No matter which material is used, or materials are used, to construct the speculum 10, the speculum 10 should be able to withstand conventional sterilization processes.

It is contemplated that most, if not all, uses of the speculum 10 will involve multiple movements of the first blade member 120 and the second blade member 210. The speculum 10 is optimally designed to facilitate these movements. In one preferred use of the invention, before the tips 122 and 212 of the first blade member 120 and the second blade member 210, respectively, are inserted into the vaginal opening, the blades 120 and 210 are brought into flush contact with each other. Flush contact may be achieved by moving the blades 120 and 210 by using two approaches. The first approach takes advantage of the ability of the linear position sliding section 350 to be slid along the track 150 of the first structure 100. Specifically, in this approach, the linear position sliding section 350 preferably is moved for the purpose of bringing the blades 120 and 210 together by first depressing its thumb pad 375 toward the handle 110 of the speculum 10 such that the angular locking clip 370 is removed from contacting the grooved ridges 130, and thereafter, while still maintaining this depression, sliding the linear position sliding section 350 along the track 150 until the first blade member 120 contacts the second blade member 210. Once the first blade member 120 is appropriately positioned, further movement of the linear position sliding section 350 may be prevented by releasing pressure upon the thumb pad 375 such that the angular locking clip 370 contacts, or “locks” within, one of the grooved ridges 130.

Bringing the blade members 120 and 210 into flush contact with each other may also require that the first blade member 120 be pivoted. This pivoting may be achieved by tilting the second blade member 210 about the axis of the hinge 220. Specifically, this pivoting preferably may be effected by first pressing the pivot position-locking arm 360 toward the second blade member 210 until it no longer contacts the notch 265 of the port 260, and thereafter, while still maintaining this upward pressure, moving the vertical pivoting arm 250 of the second structure 200 away from the handle 110. The pivot flange 270 facilitates movement of the vertical pivoting arm 250 for this purpose. Once the second blade member 210 is pivoted into a desired position with respect to the first blade member 120, the pressure applied to the locking arm 360 may be relaxed such that the notch 265 of the port 260 is held between, or “locks” between, two of the plurality of locking teeth 365.

After bringing the blades 120 and 210 together, the professional may then insert tips 122 and 212 into the vaginal opening. Once inserted, further positioning of the blades 120 and 210 may be effected by using approaches similar to those described above. For example, the blades 120 and 210 may be moved away from each other, all the while being held parallel to each other, by sliding the linear position sliding section 350 up the track 150. As with the sliding movement described above, this sliding in what is, in effect, the opposing direction, may be achieved by first depressing the thumb pad 375 in a direction toward the handle 110 to remove, or “unlock,” the angular locking clip 370 from contact with the grooved ridges 130. “Relocking” of the linear position sliding section 350, also as described before, may be achieved by releasing pressure from the thumb pad 375, thereby allowing the angular locking clip 370 to recontact, and be held by tension within, one of the grooved ridges 130.

As a second example of how proper dilation may be achieved and held, the second blade member 210 may be pivoted toward the first blade member 120 essentially as described above for pivoting the second blade member 210 away from the first blade member 120, but with one difference being that the vertical pivoting arm 250 must be moved toward, as opposed to away from, the handle 110.

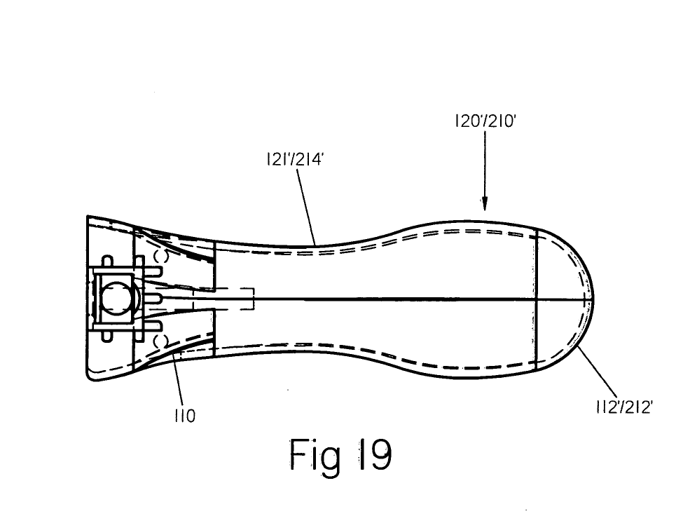

While the first blade member 120 and the second blade member 210 have been shown throughout the figures as ellipsoids of uniform dimensions through the tips 112/212 and primary sections 121/214, they may be arranged with alternative shapes. For example, as shown in FIG. 19, a second embodiment of either or both of first blade member 120′ and the second blade member 210′ may be configured with bullnose shapes such that tip 112′/212′ is wider than primary section 121′/214′. It is therefore to be understood that either or both of the first blade member 120 and the second blade member 210 may be of selectable shape, size, and/or contour. The blade members 120 and 210 may have a symmetrical or an asymmetrical shape. It is also to be noted that the blade members 120 and 210 may be of a length which is longer than, equal to, or shorter than the length of the handle 110.

It is to be understood that various modifications may be made to the device of the present invention and its methods of use without departing from the spirit and scope of the invention. For example, the steps of opening and closing the speculum 10 may be performed in differing order, one or more steps may be omitted, and one or more steps may be replaced with alternative forms thereof. Accordingly, other embodiments are within the scope of the claims appended hereto.

What is claimed is:

1. A speculum comprising:
a. a first structure including a handle and a first blade member, the first blade member including a light source holder;
b. a second structure including a second blade member; and
c. a third structure hingedly connecting the second structure to the first structure on one side thereof to enable movement of the second blade member with respect to the first blade member.

2. The speculum of claim 1 wherein the first blade member includes a tip, a primary section and a transition section, wherein the transition section defines an interface between the primary section and the handle.

3. The speculum of claim 2 wherein the transition section includes a cutaway section.

4. The speculum of claim 2 wherein a width dimension of the first blade member remains constant from the transition section to and including the tip.

5. The speculum of claim 2 wherein a width dimension of the first blade member varies from the transition section to and including the tip.

6. The speculum of claim 2 wherein the light source holder includes a transparent or open end aligned with the primary section of the first blade member.

7. The speculum of claim 6 wherein the handle includes a light source port.

8. The speculum of claim 8 wherein the light source holder extends from the light source port through to the end of the transition section.

9. The speculum of claim 2 wherein the second blade member includes a tip, a primary section, a transition section and a structural member, wherein the transition section defines an interface between the primary section and the structural member.

10. The speculum of claim 9 wherein the transition section of the second blade member includes a cutaway section.

11. The speculum of claim 9 wherein a width dimension of the second blade member remains constant from the transition section to and including the tip.

12. The speculum of claim 9 wherein a width dimension of the second blade member varies from the transition section to and including the tip.

13. The speculum of claim 1 wherein the second structure includes a pivot flange to enable hinged movement of the second blade member with respect to the first blade member.

14. The speculum of claim 1 wherein the third structure includes a linear position sliding section, a linear position-locking arm, and a pivot position-locking arm.

15. The speculum of claim 14 wherein the handle of the first structure includes a linear track and a linear position fixer, and the second structure includes a pivot flange with a retaining port.

16. The speculum of claim 15 wherein the linear position sliding section is movably engaged in the linear track, the linear position-locking arm is releasably engageable with the linear position fixer, and the pivot position-locking arm is releasably engageable with the retaining port of the pivot flange.

17. The speculum of claim 1 wherein the first structure, the second structure and the third structure are made of one or more plastics.

18. The speculum of claim 1 wherein the first structure, the second structure and the third structure are made entirely of one or more metals or metal alloys.

19. A speculum comprising:
a. a first structure having a handle, a first blade member, and a light source holder which is connected to a surface of the first blade member and is capable of holding a light source;
b. a second structure having a second blade member, a hinge which is essentially perpendicular to the second blade member, and a vertical pivoting arm which extends angularly away from the second blade member;
c. a third structure which is engaged with both the first structure and the second structure such that the first structure is indirectly engaged with the second structure, wherein the third structure has a vertical sliding arm which is slidably contained within a track in the handle, the vertical sliding arm is connected to a first vertical member having a first pin holding port and a second vertical member having a second pin holding port, and the first pin holding port holds a first pin that is associated with a first side of the hinge and the second pin holding port holds a second pin that is associated with a second side of the hinge such that both the first vertical member and the second vertical member may rotate about the axis of the hinge;
wherein sliding the vertical sliding arm in a first direction along the tract moves the second blade member away from the first blade member, and sliding the vertical sliding arm in a second direction along the tract moves the second blade member toward the first blade member; and
wherein pivoting the vertical pivoting arm away from the handle allows the second blade member to move toward the first blade member, and pivoting the vertical pivoting arm toward the handle allows the second blade member to move away from the first blade member.
